SEQUENCE LISTING (4 sequences)

SEQ ID NO: 1 - protein, 574 aa, mammalian
EDDIVIATKNGKVRGMNLTVLGGTVTAFLGIPYAQPPLGRLRFKKPQSLTKWSDIWNATKYANSCYQNIDQSFPGFHGSEMWNPNTDLSEDCLYLNVWIPAPKPKNATVMIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSSNAPWAVTSLYEARNRTLTLAKLTGCSRDNETEIVKCLRNKDPHEILLNEAFVVPYGTLLSVNFGPTMDGDFLTEMPDILLELGQFKKTQILVGVNKDEGTAFLVYGAPGFSKDNDSIITRNEFQEGLKIFFPGVSEFGKESILFHYTDWVDDQRPENYREALDDVVGDYNIICPALEFTKKFSEWGNNAFFYYFEHRSSKLPWPEWMGVMHGYEIEFVFGLPLERRVNYTKAEEILSRSIVKRWANFAKYGNPNGTHNNSTKWPVFKSTEQKYLTLNTESSRILTKLRAQQCRFWTSFFPKVLEMTGNIDEAEWEWKAGFHRWSNYMMDWKNQFNDYTSKKESCVGL

SEQ ID NO: 2 - protein, 574 aa, mammalian
EDDIVIATKNGKVRGMNLTVLGGTVTAFLGIPYAQPPLGRLRFKKPQSLTKWSDIWNATKYANSCYQNIDQSFPGFHGSEMWNPNTDLSEDCLYLNVWIPAPKPKNATVMIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSSNAPWAVTSLFEARNRTLTLAKLTGCSRENETEIVKCLRNKDPHEILLNEAFVVPYGTLLSVNFGPTMDGDFLTEMPDILLELGQFKKTQILVGVNKDEGTAFLVYGAPGFSKDNDSIITRNEFQEGLKIFFPGVSEFGKESILFHYTDWVDDQRPENYREALDDVVGDYNIICPALEFTKKFSEWGNNAFFYYFEHRSSKLPWPEWMGVMHGYEIEFVFGLPLERRVNYTKAEEILSRSIVKRWANFAKYGNPNGTHNNSTKWPVFKSTEQKYLTLNTESSRILTKLRAQQCRFWTSFFPKVLEMTGNIDEAEWEWKAGFHRWSNYMMDWKNQFNDYTSKKESCVGL

SEQ ID NO: 3 - protein, 574 aa, mammalian
EDDIVIATKNGKVRGMNLTVLGGTVTAFLGIPYAQPPLGRLRFKKPQSLTKWSDIWNATKYANSCYQNIDQSFPGFHGSEMWNPNTDLSEDCLYLNVWIPAPKPKNATVMIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSSNAPWAVTSLYEARNRTLTLAKLTGCSRENETEIIKCLRNKDPQEILLNEAFVVPYGTLLSVNFGPTMDGDFLTEMPDILLELGQFKKTQILVGVNKDEGTAFLVYGAPGFSKDNDSIITRNEFQEGLKIFFPGVSEFGKESILFHYTDWVDDQRPENYREALDDVVGDYNIICPALEFTKKFSEWGNNAFFYYFEHRSSKLPWPEWMGVMHGYEIEFVFGLPLERRVNYTKAEEILSRSIVKRWANFAKYGNPNGTHNNSTKWPVFKSTEQKYLTLNTESSRILTKLRAQQCRFWTSFFPKVLEMTGNIDEAEWEWKAGFHRWNNYMMDWKNQFNDYTSKKESCVGL

SEQ ID NO: 4 - protein, 574 aa, mammalian
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTMDGDFLTEMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

(19) United States
(12) Patent Application Publication
(10) Pub. No.: US 2007/0134205 A1
(43) Pub. Date: Jun. 14, 2007

(54) Gene delivery of detoxifying agent

(75) Inventor: Yvonne Rosenberg, Washington, DC (US)

Correspondence Address: BELL, BOYD, & LLOYD LLP, P.O. BOX 1135, CHICAGO, IL, 60690, US

(73) Assignee: Procell, Rockville, MD (US)

(21) Appl. No.: 11/435,196

(22) Filed: May 16, 2006

Related U.S. Application Data
(60) Provisional application No. 60/681,826, filed on May 16, 2005.

(51) Int. Cl. A61K 48/00; C12N 15/86

(52) U.S. Cl. 424/93.2; 435/456; 977/802

(57) ABSTRACT

Gene delivery of a detoxifying agent is described.

### BACKGROUND OF THE INVENTION

The role of acetylcholinesterase (AChE) is to hydrolyse the neurotransmitter, acetylcholine, at neuromuscular junctions and other cholinergic. synapses, thereby limiting repetitive neuronal stimulation which may result in convulsions, respiratory failure and death (1, 2). Compounds capable of inhibiting AChE activity, such as those containing carboxylic or phosphoric esters (organophosphates, OP), result in accumulation of acetylcholine and potentially serve as potent neurotoxins; posing threats in both military and civilian arenas.

The military programs, as mandated in DTO CB32 and DTO D, seek to develop alternate delivery methods for recombinant (r) protein vaccines and nerve agent scavengers. These include evaluation of respiratory vaccination and delivery of bioscavengers, such as recombinant human (Hu) butyrylcholinesterase (BChE) (rHuBChE) via gene therapy, such that pretreatment with HuBChE can detoxify nerve agents at a sufficient rate to protect the warfighter from exposure of up to 5×LD₅₀ of nerve agents without physiological side effects.

Traditional treatment for poisoning by OP compounds consists of a combination of drugs such as carbamates, antimuscarinics, reactivators of inhibited AChE and anti-convulsants in pre-and postexposure modalities (1, 2). However, while this approach results in preventing fatal effects of OP toxicity, it is far from optimal since the best pretreatment/therapy regime, i.e., pyridostigmine pretreatment and atropine/oxime reactivator and anti-convulsant drug therapy, does not prevent severe post exposure convulsions, respiratory distress, tremors, unconsciousness (6, 7) and behavioral impairments (6, 8).

While many different catalytic and stoichiometric enzymes from various species exhibit the ability to detoxify neurotoxins (9-13), based on availability, broad spectrum efficacy, stability and safety, homologous native BChE is a suitable best candidate in terms of developing a human treatment (1). BChE (EC 3.1.1.8, acylcholine anhydrolase pseudocholinesterase, non-specific cholinesterase) is a serine esterase (MW=345,000) comprised of four identical subunits containing 574 amino acids, held together by non-covalent bonds, and 36 carbohydrate chains (23.9% by weight) (14). Sialic acid content plays a critical role in the circulatory half-life of cholinesterases (ChE) since exposed galactose residues rapidly bind asialoreceptors in the liver and accelerate elimination of enzyme (15, 16). Human BChE is encoded by a single gene (the active site: serine 198), with monkey BChE sharing 95.6% identity with the human enzyme and differing in only 23 amino acids. At the protein level, primate BChE is present predominantly in plasma, leading to relative ease of production, while AChE is found in red blood cell membranes and in nerve synapses and thus is more difficult to purify. Recent studies using AChE knockout mice suggest that BChE might also be a physiologically important target for OP agents (17).

Currently, outdated and unused human plasma and other blood byproducts such as BChE containing Cohn Fraction IV-4 are a ready and large source of HuBChE. However, due to batch to batch variability, cost, limits to plasma availability and potential safety issues associated with increased contamination of human blood with agents such as hepatitis, HIV, prions, West Nile virus and so on, alternate strategies for the in vivo administration of BChE are required.

Exogenous BChE can be effectively delivered by im and iv routes although the latter takes much longer to reach peak levels in the circulation (˜1 hr vs 12 hr) (1).

The hallmark of the highly stable plasma-derived bioscavenger molecule is its long circulatory retention time and lack of immunogenicity. The critical features determining stability of BChE molecules are the preservation of the native glycosylation patterns, efficient tetramerization and the absence of antigenic epitopes. However, unlike the native forms of BChE, which consist predominantly of complex bi-antennary types of glycan structures, rBChE molecules contain glycans that display a wide heterogeneity and lack the mature glycans that characterize the major constituents of native enzymes (15, 16, 18). In addition, rHuBCbE is primarily expressed as a mixture of monomers and dimers with only 10-30% tetramers (19), compared to the stable native plasma derived enzyme that is >95% tetrameric in form (18). Thus, without additional post-translational modifications e.g. in vitro glycosylation or PEGylation, recombinant technology has not yet provided a viable BChE bioscavenger treatment alternative to the native enzyme. In this context, gene therapy may overcome such stability issues evident with rBChE since the sugar profiles and the process of tetramerization will be performed in vivo and should mimic the native molecule.

With the continuing development of DNA recombinant technology, gene therapy is emerging as a promising approach to achieve high-level in vivo expression of potential vaccines as well as of therapeutic proteins that are very similar to their native counterparts. Many viral based vectors, such as, adenovirus, retrovirus, adeno-associated virus (AAV), vaccinia virus, and lentiviruses, such as, HIV and BIV have been developed as delivery vehicles for vaccines against cancers, infectious disease and genetic disorders.

### SUMMARY OF THE INVENTION

The instant invention relates to the use of gene therapy systems, such as, viral vector systems, to deliver detoxifying agents to a host.

A suitable viral vector is an adeno-associated viral (AAV) vector. A particularly preferred AAV vector is one which targets those tissues most likely to be exposed to an OP, such as the lungs and skin. A suitable detoxifying agent is an acetylcholinesterase. A preferred enzyme is butyrylcholinesterase. Any polypeptide with suitable OP detoxifying activity and any polynucleotide encoding such a polypeptide can be cloned in a vector of interest.

Further aspects of the invention of interest are provided hereinbelow.

### BRIEF DESCRIPTION OF THE DRAWINGS

FIG. 1 depicts plasmids.

FIG. 2 compares amino acid sequences.

FIG. 3 is a construct.

FIG. 4 depicts expression of monomer and tetramer.

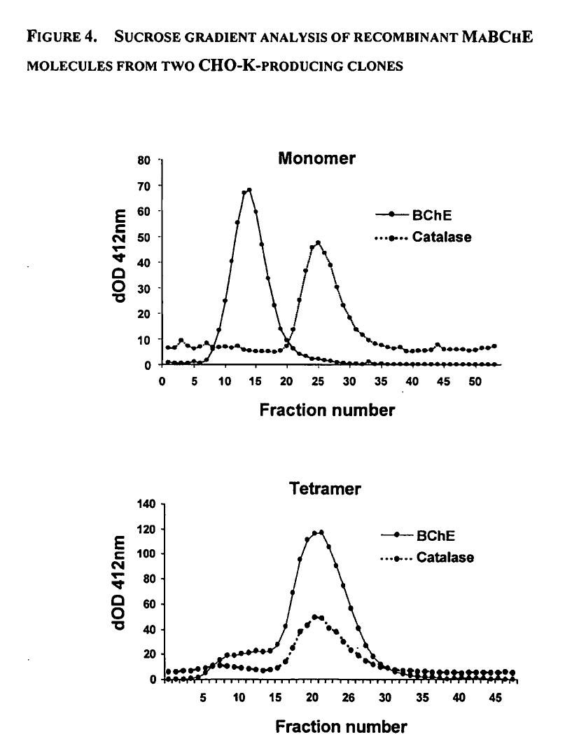

### DETAILED DESCRIPTION OF THE INVENTION

For the purposes of the instant invention, a detoxifying agent is one which inactivates or disables the deleterious effects of a compound in a host. One category of detoxifying agent is a bioscavenger, a molecule that is biologically compatible and which counteracts the biologically negative effects of a toxic agent. That can occur by binding and sequestering the toxic agent, chemically modifying the toxic agent removing the deleterious activity and so on.

A number of toxic agents are known, for example, those of biological origin, such as microorganisms, toxoids and toxins produced by microorganisms, plants and animals, such as tetrodotoxin, botulinum toxin, snake venom, conotoxin, tropane alkaloids and so on, as well as synthetic compounds, such as nerve agents, such as, VX (ethyl-S-2-diisopropylaminoethyl-phosphano-thiolate), MEPQ (7-(methylethoxy-phosphinyloxy)-1-methylquinolinium iodide), soman (pinacolyl methlphosphonofluoridate), DFP (diisopylfluorophosphate), VG and so on, as well as insecticides, herbicides, other organophosphates and so on. Other toxic agents include recreational drugs, particularly those which are habituating, such as heroin and cocaine. Yet other toxic agents include drugs that have untoward side effects, such as apnea or paralysis arising from exposure to succinylcholine. The instant invention relates to a method that renders said toxic agent ineffective, which, for the purposes of the instant invention, is described as inactivating said toxic agent.

Suitable such detoxifying agents, or bioscavengers, include those which specifically or non-specifically bind and sequester a toxic agent, as well as those that inactivate a toxic agent. Examples of the latter class of molecules of interest include butyrylcholinesterase (BChE), acetycholinesterase (AChE), organophosphate hydrolase (OPH), organophophorous acid anhydride hydrolase (OPAA), parathion hydrolase, paraoxonase and carboxylesterase. The instant invention contemplates the delivery of an intact molecule on interest, truncated forms thereof, monomers thereof, subunits thereof, variants thereof and so on, that retain the biological function of interest, namely, a detoxifying activity. In the discussion to follow, BChE is exemplified, however, the materials and methods of the instant invention can be practiced with any detoxifying agent.

Viral vector mediated gene therapy based approaches to introduce HuBChE as an in vivo antidote offer several advantages over the administration of recombinant or native BChE proteins. (i) While each of these comes in contact with and inactivates circulating OPs, BChE introduced via a suitable viral vector may inactivate OP both in the circulation and also at the site of injury i.e. at the neuromuscularjunctions. (ii) In contrast to native BChE, which has a half life of 8-12 days (3,4), a BChE gene introduced in vivo using a viral vector based gene delivery platform can express the transgene, essentially, indefinitely, and certainly, up to several months. (iii) Such genes may be administered as an aerosol, as in cystic fibrosis treatment (5) or adapted for non-invasive pulmonary (inhaler) delivery for the war fighter and civilian populations.

Conventional molecular biology, microbiology, and recombinant DNA techniques within the skill of the art are practiced, for example, Sambrook et al., 1989, Molecular cloning a laboratory manual. 2ed. Cold Spring Harbor Laboratory, Cold spring Harbor, N.Y.; Glover, 1985, DNA Cloning: A Practical Approach, vols. I and II, Oligonucleotide Synthesis, MRL Press, LTD., Oxford, U.K.; Hames and Higgins, 1985, Transcription and Translation; Hames and Higgins, 1984, Animal Cell Culture; Freshney, 1986, lrnmobilized Cells And Enzymes, IRL Press; and Perbal, 1984, A Practical Guide to Molecular Cloning.

The terms “nucleic acid”, “polynucleotide”, “oligonucleotide” or “nucleotide sequence” cover RNA, DNA, or cDNA sequences or alternatively RNA/DNA hybrid sequences of more than one nucleotide, either in the single-stranded form or in the duplex, double-stranded form.

“Nucleotide” relates to the natural nucleotides as well as a purine analog, a pyrimidine analog or a sugar compound that carries a purine or pyrimidine base, or any molecule which can hybridize to a base in a nucleic acid or to a nucleic acid.

The term “homologous” refers to nucleic acid or proteins that possess a “common evolutionary origin,” including proteins from superfamilies (e.g., the immunoglobulin superfamily) and homologous proteins from different species (e.g., myosin light chain, etc.) (Reeck et al., 1987, Cell 50:667)). Such proteins (and their encoding genes) have sequence homology.

Accordingly, the term “sequence similarity” refers to the degree of identity or correspondence between nucleic acid or amino acid sequences of proteins that may or may not share a common evolutionary origin (see Reeck et al., supra).

The term “equivalent amino acid residues” herein means the amino acids occupy substantially the same position within a protein sequence when two or more sequences are aligned for analysis. Preferred BChE polypeptides of the instant invention have an amino acid sequence sufficiently identical to that of the wild-type BChE.

By “wild-type” is meant the most prevalent form or allele present in a defined population, whether local or wider in scope.

The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having about 55% identity, preferably 65% identity, more preferably 75%, 85%, 95% or 98% identity, with BChE activity are defined herein as sufficiently identical. The similar or homologous sequences can be identified by alignment using, for example, the GCG (Genetics Computer Group, Program Manual for the GCG Package, Version 7, Madison, Wis.) pileup program.

DNA sequences are “substantially homologous” or “substantially similar” when at least about 50% (preferably at least about 75%, and more preferably at least about 90 or 95%) of the nucleotides match over the defined length of the DNA sequences. Sequences that are substantially homologous can be identified practicing methods known in the art, e.g., Maniatis et al., supra; Glover et al. supra.; and Hames and Higgins, supra. The invention further encompasses nucleic acid molecules that differ from the nucleotide sequence of the disclosed BChE due to degeneracy of the genetic code and thus encode substantially the same BChE protein as that previously disclosed.

A gene encoding a BChE polypeptide of the invention, whether genomic DNA or cDNA, can be isolated from any source, such as a human cell or cDNA or genomic library. Methods for obtaining genes are well known in the art. Accordingly, any animal cell potentially can serve as the nucleic acid source for the molecular cloning of a BChE gene.

“Variant” of a nucleic acid or polypeptide according to the invention will be understood to mean a nucleic acid or polypeptide which differs by one or more bases or amino acids relative to the reference polynucleotide or polypeptide. A variant nucleic acid or polypeptide may be of natural origin, such as an allelic variant which exists naturally, or it may also be a nonnatural variant obtained, for example, by mutagenic techniques. Advantageous nucleic acid variants are those that yield polypeptides with desired features, such as enhanced half-life in a host, enhanced detoxifying ability and so on.

It will be appreciated by those skilled in the art that DNA sequence polymorphisms that lead to changes in the amino acid sequences of BChE may exist within a population (e.g., the human population). Such genetic polymorphism in the BChE coding sequence may exist among individuals within a population due to natural allelic variation. An allele is one of a group of genes that occur alternatively at a given genetic locus.

As used herein, the terms “gene” and “recombinant gene” refer to nucleic acid molecules comprising an open reading frame encoding, for example, a BChE protein, preferably a mammalian BChE protein.

As used herein, the phrase “allelic variant” refers to a nucleotide sequence that occurs at a, in the case of BChE, at a BChE locus or to a polypeptide encoded by the nucleotide sequence. Alternative alleles can be identified by sequencing the gene of interest in a number of different individuals. That can be carried out readily by using hybridization probes to identify the same genetic locus in a variety of individuals. Any and all such nucleotide variations and resulting amino acid polymorphisms or variations in BChE that are the result of natural allelic variation and that do not alter the functional activity of BChE are intended to be within the scope of the invention. Moreover, nucleic acid molecules encoding BChE proteins from other species (BChE homologues) with a nucleotide sequence that differs from that of a human BChE but have substantially the same activity, are intended to be within the scope of the invention. Nucleic acid molecules corresponding to natural allelic variants and homologues of the BChE cDNA of the invention can be isolated based on identity with the human BChE nucleic acids disclosed herein using the human CDNA or a portion thereof, as a hybridization probe according to standard hybridization techniques under stringent hybridization conditions.

Conditions of temperature and ionic strength determine the “stringency” of hybridization between nucleic acids. For preliminary screening for homologous nucleic acids, low stringency hybridization conditions, corresponding to, for example, a Tₘ of 55°. High stringency hybridization conditions correspond to higher, Tₘ, e.g., 50% formamide, 5× or 6×SSC.

A variant of a polypeptide includes a polypeptide whose amino acid sequence contains one or more substitutions, additions or deletions of at least one amino acid residue, relative to the amino acid sequence of the reference polypeptide, and the amino acid substitutions may be either conservative or nonconservative. A variant of a polypeptide or protein is any analogue, fragment, derivative, or mutant which is derived from a polypeptide or protein and which retains at least one biological property of the polypeptide or protein. Different variants of the polypeptide or protein may exist in nature. These variants may be allelic variations characterized by differences in the nucleotide sequences of the structural gene coding for the protein, or may involve differential splicing or post-translational modification. Variants also include a related protein having substantially the same biological activity, but obtained from a different species. In addition to naturally-occurring allelic variants of the BChE sequence that may exist in the population, the skilled artisan will further appreciate that changes can be introduced by mutation into the nucleotide sequence, thereby leading to changes in the amino acid sequence of the encoded BChE, without substantially altering the biological activity of the BChE protein. Thus, one can make nucleotide substitutions leading to amino acid substitutions at “non-essential” amino acid residues.

A “non-essential” amino acid residue is a residue that can be altered from the wild type sequence of BChE without substantially altering the biological activity.

An “essential” amino acid residue is one required for substantial biological activity. For example, amino acid residues that are not conserved or only semi-conserved among BChE of various species may be non-essential for activity and thus would be likely targets of alteration. Alternatively, amino acid residues that are conserved among the BChE proteins of various species may be essential for activity and thus would not be likely targets for alteration.

As used interchangeably herein, a “detoxifying activity”, “biological activity of a detoxifying agent” or “functional activity of a detoxifying agent”, refers to an activity exerted by a detoxifying agent, such as a protein, polypeptide or nucleic acid molecule of a detoxifying agent expressing cell as determined in vivo or in vitro, according to standard techniques. A BChE activity can be a direct activity, such as an enzymatic activity on a second protein or an indirect activity, such as a cellular signaling activity mediated by interaction of BChE with a second protein.

The skilled artisan can produce variants having single or multiple amino acid substitutions, deletions, additions, or replacements. These variants may include, inter alia: (a) variants in which one or more amino acid residues are substituted with conservative or non-conservative amino acids, (b) variants in which one or more amino acids are added to the polypeptide or protein, (c) variants in which one or more of the amino acids includes a substituent group, and (d) variants in which the polypeptide or protein is fused with another polypeptide such as serum albumin. The techniques for obtaining these variants, including genetic (suppressions, deletions, mutations, etc.), chemical, and enzymatic techniques, are known to persons having ordinary skill in the art.

If such allelic variations, analogues, fragments, derivatives, mutants, and modifications, including alternative MRNA splicing forms and alternative post-translational modification forms result in derivatives of the polypeptide which retain any of the biological properties of the polypeptide, they are intended to be included within the scope of this invention.

Another aspect of the invention pertains to vectors, preferably expression vectors, containing a nucleic acid encoding BChE (or a portion thereof). As used herein, the term “vector” refers to a nucleic acid molecule capable of transporting another nucleic acid linked thereto. One type of vector is a “plasmid” that refers to a circular double-stranded DNA loop into which additional DNA segments can be ligated. Another type of vector is a viral vector, wherein additional DNA segments can be ligated into a viral genome. Certain vectors are capable of autonomous replication in a host cell (e.g., bacterial vectors having a bacterial origin of replication and episomal mammalian vectors). Other vectors (e.g., non-episomal mammalian vectors) are integrated into the genome of a host cell on introduction into the host cell and thereby are replicated along with the host genome. Moreover, certain vectors, expression vectors, are capable of directing the expression of genes operably linked thereto. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), that serve equivalent functions, as well as non-viral vectors, such as synthetic chemicals for carrying a nucleic acid.

The recombinant expression vectors of the invention comprise a nucleic acid of the invention in a form suitable for expression of the nucleic acid in a host cell. That means the recombinant expression vectors include one or more regulatory sequences, selected on the basis of the host cells to be used for expression, which is linked operably to the nucleic acid to be expressed.

Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The recombinant expression vectors of the invention can be designed for expression of a detoxifying agent in prokaryotic or eukaryotic cells, e.g., bacterial cells such as E. coli, insect cells (using baculovirus expression vectors), yeast cells or mammalian cells. Suitable host cells are discussed further in Goeddel, infra. Alternatively, the recombinant expression vector can be transcribed and translated in vitro, for example using T7 promoter regulatory sequences and T7 polymerase.

In another embodiment, the expression vector is a yeast expression vector. Examples of vectors for expression in yeast such as S. cerevisiae include pYepSecl (Baldari et al., EMBO J. (1987) 6:229-234), pMFa (Kurjan et al., Cell (1982) 30:933-943), pJRY88 (Schultz et al., Gene (1987) 54:113-123), pYES2 (Invitrogen Corporation, San Diego, Calif.) and pPicZ (Invitrogen Corp, San Diego, Calif.).

Alternatively, a detoxifying agent can be expressed in insect cells using baculovirus expression vectors. Baculovirus vectors available for expression of proteins in cultured insect cells (e.g., Sf 9 cells) include the pAc series (Smith et al., Mol. Cell. Biol. (1983) 3:2156-2165) and the pVL series (Lucklow et al., Virology (1989) 170:31-39).

In yet another embodiment, a nucleic acid of the invention is expressed in mammalian cells using a mammalian expression vector. Examples of mammalian expression vectors include pCDM8 (Seed, Nature (1987) 329:840) and pMT2PC (Kaufrnan et al., EMBO J. (1987) 6:187-195). When used in mammalian cells, the control functions of the expression vector often are provided by viral regulatory elements. For example, commonly used promoters are derived from polyoma, adenovirus 2, cytomegalovirus and simian virus 40. For other suitable expression systems for both prokaryotic and eukaryotic cells, see chapters 16 and 17 of Sambrook et al., supra.

For stable transformation of mammalian cells, to identify and to select the integrants, a gene that encodes a selectable marker (e.g., for resistance to antibiotics) generally is introduced into the host cells along with the gene of interest. Preferred selectable markers include those that confer resistance to drugs, such as G418, hygromycin and methotrexate. Nucleic acid encoding a selectable marker can be introduced into a host cell on the same vector as that encoding a detoxifying agent or can be introduced on a separate vector. Cells stably transfected with the introduced nucleic acid can be identified by drug selection (e.g., cells that have incorporated the selectable marker gene will survive, while the other cells die). Often, mammal cells are suitable host cells as those cells more likely mimic the naturally occurring post translational modification observed for the native, wild-type molecule, such as proper glycosylation at the appropriate sites in the molecule.

Expression control sequences or regulatory sequences regulate the expression of a nucleic acid. A regulatory region may include sequences which are naturally responsible for expressing a particular nucleic acid (a homologous region) or may include sequences of a different origin (heterologous, responsible for expressing different proteins or even synthetic proteins). In particular, the sequences can be sequences of eukaryotic or viral genes or derived sequences which stimulate or repress transcription of a gene in a specific or non-specific manner and in an inducible or non-inducible manner. Regulatory regions include origins of replication, RNA splice sites, enhancers, transcriptional termination sequences, signal sequences which direct the polypeptide into the secretory pathways of the target cell, promoters, polyA sites and the like. Goeddel, Gene Expression Technology: Methods in Enzymology Vol. 185, Academic Press, San Diego, Calif. (1990).

A “promoter sequence” initiates transcription of a downstream (3′ direction) coding sequence. For purposes of defining the present invention, the promoter sequence generally is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. The promoter can be tissue-specific, constitutive, inducible and so on.

A “signal sequence” directs the host cell to translocate the polypeptide, generally for secretion out of the cell.

“Homology” means similarity of sequence reflecting a common evolutionary origin. Polypeptides or proteins are said to have homology, or similarity, if a substantial number of their amino acids are either (1) identical, or (2) have a chemically similar R side chain. Nucleic acids are said to have homology if a substantial number of their nucleotides are identical.

A “vector” is a replicon, such as plasmid, virus, phage or cosmid, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control.

The present invention also relates to cloning vectors containing genes encoding analogs and derivatives of any of the detoxifying agents, such as, BChE polypeptide of the invention, that have the same or homologous functional activity as that of BChE polypeptide, and homologs thereof from other species.

A wide variety of host/expression vector combinations are available, such as derivatives of SV40 and known bacterial plasmids, e.g., Escherichia coli plasmids col E1, pCR1, pBR322, pMal-C2, pET, pGEX (Smith et al., 1988, Gene, 67:31-40), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences; and the like.

For example, in a baculovirus expression systems, both non-fusion transfer vectors, such as but not limited to pVL941 (BamHI cloning site; Summers), pVL1393 (BamH1, SmaI, XbaI, EcoRI, NotI, XrnaIII, BglII, and PstI cloning site; Invitrogen), pVL1392 (BglII, PstI, NotI, XmaIII, EcoRI, XbaI, SmaI, and BamH1 cloning site; Summers and Invitrogen), and pBlueBacIII (BamHI, BglII, PstI, NcoI, and HindIII cloning site, with blue/white recombinant screening possible; Invitrogen), and fusion transfer vectors, such as, but not limited to pAc700 (BamHI and KpnI cloning site, in which the BamHI recognition site begins with the initiation codon; Summers), pAc701 and pAc702 (same as pAc700, with different reading frames), pAc360 (BamHI cloning site 36 base pairs downstream of a polyhedrin initiation codon; Invitrogen(195)), and pBlueBacHisA, B, C (three different reading frames, with BamHI, BglII, PstI, NcoI, and HindIII cloning site, an N-terminal peptide for ProBond purification, and blue/white recombinant screening of plaques can be used.

Yeast expression systems can also be used according to the invention to express a BChE polypeptide. For example, the non-fusion pYES2 vector (XbaI, SphI, ShoI, NotI, GstXI, EcoRI, BstXI, BamHI, SacI, KpnI, and HindIII cloning sit; Invitrogen) or the fusion pYESHisA, B, C (XbaI, SphI, ShoI, NotI, BstXI, EcoRI, BamH1, SacI, KpnI, and HindIII cloning site, N-terminal peptide purified with ProBond resin and cleaved with enterokinase; Invitrogen), to mention just two, can be employed according to the invention.

Vectors are introduced into the desired host cells by methods known in the art, for example, transfection, electroporation, microinjection, transduction, cell fusion, DEAE dextran, calcium phosphate precipitation, lipofection (lysosome fusion), use of a gene gun, or a DNA vector transporter (Wu et al., 1992, J. Biol. Chem., 267:963-967; and Wu & Wu, 1988, J. Biol. Chem., 263:14621-14624; Hartmut et al., supra).

The invention contemplates delivery of a vector that will express a therapeutically effective amount of any one detoxifying agent, such as a BChE polypeptide, for gene therapy applications. The phrase “therapeutically effective amount” is used herein to mean an amount sufficient to reduce by at least about 15 percent, preferably by at least 50 percent, more preferably by at least 90 percent, and still more preferably prevent, a clinically significant deficit in the activity, function and response of the host. Alternatively, a therapeutically effective amount is sufficient to cause an improvement in a clinically significant condition in the host. In the context of detoxifying agents, often the level of effectiveness is defined in terms of protecting/scavenging/detoxifying a certain amount of toxic agent. A useful metric is LD₅₀, which is the amount of chemical that is lethal to one half of the animals exposed to the chemical. Thus, a therapeutically effective amount also is one which is sufficient to protect at least one LD₅₀ of toxic agent. Certainly, it would be advantageous if the invention of interest can deliver amounts of detoxifying agent that protect, 2, 3, 4, 5 or 6 times or more of LD₅₀ of toxic agent.

Adenoviral vectors such as the human adenovirus of type 2 or 5 may be used as vectors. Various serotypes, whose structure and properties vary somewhat, have been characterized. Among these serotypes, human adenoviruses of type 2 or 5 (Ad 2 or Ad 5) or adenoviruses of animal origin (see Application WO 94/26914) can be used in the context of the present invention. Among the adenoviruses of animal origin which can be used in the context of the present invention, there may be mentioned adenoviruses of canine, bovine, murine (example: Mav1, Beard et al., Virology 75 (1990) 81), ovine, porcine, avian or simian (example: SAV) origin. Preferably, the defective adenoviruses of the invention comprise the ITRs, a sequence allowing the encapsidation and the sequence encoding a BChE protein. Preferably, in the genome of the adenoviruses of the invention, the E1 region at least is made nonfunctional. Still more preferably, in the genome of the adenoviruses of the invention, the E1 gene and at least one of the E2, E4 and L1-L5 genes are nonfunctional. The viral gene considered may be made nonfunctional by any technique known to a person skilled in the art, and in particular by total suppression, by substitution, by partial deletion or by addition of one or more bases in the gene(s) considered. Such modifications may be obtained in vitro (on the isolated DNA) or in situ, for example, by means of genetic engineering techniques, or by treatment by means of mutagenic agents. Other regions may also be modified, and in particular the E3 (WO95/02697), E2 (WO94/28938), E4 (WO94/28152, WO94/12649, WO95/02697) and L5 (WO95/02697) region. The adenovirus can have a deletion in the E1 and E4 regions and the sequence encoding a BChE is inserted at the level of the inactivated E1 region.

The defective recombinant adenoviruses according to the invention may be prepared by any technique known to persons skilled in the art (Levrero et al., 1991 Gene 101; EP 185 573; and Graham, 1984, EMBO J., 3:2917). In particular, they may be prepared by homologous recombination between an adenovirus and a plasrnid carrying, inter alia, the nucleic acid encoding the short or full length BChE. The homologous recombination occurs after cotransfection of said adenoviruses and plasmid into an appropriate cell line. The cell line used must preferably (i) be transformable by said elements, and (ii), contain the sequences capable of complementing the part of the defective adenovirus genome, preferably in integrated form in order to avoid the risks of recombination. By way of example of a line, there may be mentioned the human embryonic kidney line 293 (Graham et al., 1977, J. Gen. Virol., 36:59), which contains in particular, integrated into its genome, the left part of the genome of an Ad5 adenovirus (12%) or lines capable of complementing the E1 and E4 functions.

Herpesviruses, lentiviruses and retroviruses can be used as vectors, the construction of recombinant vectors has been widely described in the literature, see, in particular Breakfield et al., (1991, New Biologist 3:203); EP 453242; EP178220; (1985); McCormick, (1985. BioTechnology 3:689) and the like.

AAV is a human parvovirus and is one of the smallest (20-30 nm) and structurally simplest of the DNA viruses; lacking replication competency which is restored only with helper viruses such as adenovirus, herpesvirus, or vaccinia (20). In the absence of the helper functions, AAV becomes latent in mammalian cells. AAV contains a ˜4.7 kb ssDNA genome which holds two genes, rep and cap, between two inverted terminal repeats (ITRs). Non-structural rep-encoded proteins interact with the ITR sequences in a site-specific and strand-specific manner; the ITR sequences containing all the cis-acting sequences for packaging, replication, and integration. The cap gene encodes three structural capsid proteins, VPI, VP2, and VP3. Each AAV particles consists of total 60 capsid proteins forming a icosahedral structure. Nine AAV serotypes (AAVI-AAV9) have been isolated from human or nonhuman primates and fully characterized with respect to nucleotide sequence (rev in 21,22). AAV5 is quite dissimilar to the others, with a distinct ITR structure and only 67% homology of the rep gene to that of AAV2 which is the most extensively studied and developed as a vector for gene transfer in vitro and in vivo.

AAV-based gene delivery vectors (23) are increasingly used as delivery vehicles for achieving stable gene expression in both clinical and preclinical studies because of (i) the inherent stability of the vector genome with very little risk of insertional mutagenesis and activation of oncogenes (ii) the longevity (up to 18 months) of transgene expression in vivo, (iii) lack of pathogenicity of wild-type AAV in humans, (iv) their wide tropism (vi) their transducibility of both dividing and quiescent cells and (vii) an absence of inflammation induced by AAV capsid protein to date (viii) the number ongoing clinical trials using the rAAV vector gene delivery system.

Several methods are currently in use for the large-scale production of rAAV using packaging cell lines. Simplified methods have been developed for generating rAAV based on stable HeLa cell lines or HEK 293 cell lines containing integrated copies of the AAV rep and cap genes and a rAAV vector. To generate rAAV, the cells are simply infected with wild-type adenovirus, which results in induction of the integrated p5 rep promoter which initiates rAAV synthesis. rAAV producer cell lines are isolated by first cloning the desired cDNA sequence into an expression cassette that is flanked by AAV ITR. Also present on this plasmid is a selectable marker gene (neomycin resistance) and the native rep2-capX helper genes. Following HeLa cell transfection, stable cell lines are isolated and screened for viral production by adenovirus infection and yields quantified by Q-PCR or dot blot hybridization. Depending on the producer cell line, viral yields of between 5×10³×5×10⁴ DNA containing particles per cell are typical. Importantly, recombinant virus generated in this manner appears free of replication competent wild type AAV (<1 IU of wild type AAV/10¹¹ rAAV particles). As with all stable cell line production methods, the process is amenable to various methods of high-density cell growth. More than 10¹⁰ cells per production run are routinely grown using a mid-scale adherent cell bioreactor (Corning Cell Cube). Assuming a particle per cell yield of 10⁴, this stable cell line approach can routinely yield between 10¹⁴ rAAV particles per large-scale preparation.

Despite these many useful properties, AAV vectors also have limitations. AAVs were initially found as contaminating entities during the isolation of adenovirus and 50-80% of the human population are seropositive for AAV2 capsid proteins (24) making repeated administration of an AAV2 vector problematic leading to greatly attenuated immune responses in the case of vaccines (25-27). The limitations can be overcome through the development of vectors based on other serotypes, from non-human primates (e.g. AAV7, AAV8 and humans AAV-1, AAV5, AAV9) which enter via cell receptors distinct from AAV2, are not commonly found in humans, are not cross-reactive and thus would not boost anti-AAV-2 antibody production and eliminate the vector. This easy alteration of tropism by transcapsidation of AAV2 using different AAV serotype (cross dressing the virion) permits not only successful gene transfer in the setting of preexisting neutralizing antibodies as well as broadening the tropism of the vectors.

With respect to the production of BChE by gene delivery, AAV2 can be packaged with the AAV9 capsid to create a single vector that has tropism for both lung and muscle cells (40). Human AAV9 has recently been described and characterized (28). Otherwise, both AAV2/5 and AAV2/1 vectors would have to be generated to test lung cells and muscle cells.

AAV2-based vectors have exhibited an impressive longevity of transgene expression following single or repeated transduction of muscle (29), CNS (30), retina (31), liver (32), neurons (33), airway epithelial cells (34) and haemopoietic stem cells (35). For airway epithelial cells, AAV5 or AAV9 transfer is >50-fold efficient, partly due to the use of different receptors than AAV2 (36, 37). Similarly either AAV9 or AAV1 are optimal for infection of muscle cells. Importantly, the mouse C2C12 muscle line that was selected for gene transfer, can form muscle fibres in vitro and with the addition of motor neurons can form neuromuscular junctions in vitro, thus permitting the study of in vitro production/secretion of BChE in the junction and possible interactions with toxins.

There are ongoing trials with both rAAV2-based aerosol-mediated CFTR gene delivery cystic fibrosis patients (34) and rAAV2 carrying human α₁-antitrypsin gene delivery via im (38). These preclinical studies and clinical trials lend support to the use of a vector of interest carrying a gene of interest as a prophylactic treatment against a toxic agent.

Non-viral delivery of a nucleic acid encoding a detoxifying agent of interest also are contemplated in the practice of the instant invention. Thus, liposomes, lipoidal molecules, such as cationic lipids, as known in the art can be used. Also, the gene itself, known as naked DNA, also can be used.

“Pharmaceutically acceptable vehicle or excipient ” includes diluents and fillers which are pharmaceutically acceptable for method of administration, are sterile, and may be aqueous or oleaginous suspensions formulated using suitable dispersing or wetting agents and suspending agents. The particular pharmaceutically acceptable carrier and the ratio of active compound to carrier are determined by the solubility and chemical properties of the composition, the particular mode of administration, and standard pharmaceutical practice.

Any nucleic acid, polypeptide, vector, or host cell of the invention will preferably be introduced in vivo in a pharmaceutically acceptable vehicle or excipient. The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “excipient” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as excipients, particularly for injectable solutions. Suitable pharmaceutical excipients are described in “Remington's Pharmaceutical Sciences” by E. W. Martin.

A pharmaceutical composition of the invention is formulated to be compatible with the intended route of administration. Examples of routes of administration include parenteral, e.g., intravenous, intradermal, intramuscular, intraperitoneal, subcutaneous, oral (e.g., inhalation), transdermal (topical), transmucosal and rectal administration. Solutions or suspensions used for parenteral, intradermal or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose. pH can be adjusted with acids or bases, such as HCl or NaOH. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL® (BASF; Parsippany, N.J.) or phosphate-buffered saline (PBS). In all cases, the composition must be sterile and should be fluid to the extent that easy syringability exists. The composition must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyetheylene glycol and the like) and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols such as mannitol, sorbitol or sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of the active ingredient plus any additional desired ingredient from a previously sterile-filtered solution thereof.

Oral compositions generally include an inert diluent or an edible carrier. The compositions can be enclosed in gelatin capsules or compressed into tablets. For the purpose of oral therapeutic administration, the active compound can be incorporated with excipients and used in the form of tablets, troches or capsules. Oral compositions also can be prepared using a fluid carrier to yield a syrup or liquid formulation, or for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate or orange flavoring.

For administration by inhalation, the compounds are delivered in the form of an aerosol spray from a pressurized container or dispenser that contains a suitable propellant, e.g., a gas such as carbon dioxide or a nebulizer.

Systemic administration also can be by transmucosal or transdermal means. For transmucosal or transdermal administration, penetrants appropriate to the barrier to be permeated are used in the formulation. Such penetrants generally are known in the art and include, for example, for transmucosal administration, detergents, bile salts and fusidic acid derivatives. Transmucosal administration can be accomplished through the use of nasal sprays or suppositories. For transdermal administration, the active compounds are formulated into ointments, salves, gels or creams as generally known in the art, or delivered by a patch adhered to the skin.

The compounds also can be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid.

Methods for preparation of such formulations will be apparent to those skilled in the art. The materials also can be obtained commercially from Alza Corporation and Nova Pharmaceuticals, Inc. Liposomal suspensions (including liposomes targeted to infected cells with monoclonal antibodies) also can be used as pharmaceutically acceptable carriers. Those can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. Depending on the type and severity of the disease, about 1 μg/kg to 15 mg/kg (e.g., 0.1 to 20 mg/kg) of active ingredient is an initial candidate dosage for administration to the patient, whether, for example, by one or more separate administrations or by continuous infusion. A typical daily dosage might range from about 1 μg/kg to 100 mg/kg or more, depending on the factors mentioned above. Other suitable hosts include animals, such as pets, such as cats and dogs, and livestock, including cattle, sheep, pigs and the like. The dose also can be delivered by way of edible foods, such as through transgenic plants or in an edible product of a transgenic animal, such as milk carrying a molecule of interest, as known in the art, for example. For repeated administrations over several days or longer, depending on the condition, the treatment is sustained until a desired suppression of disease symptoms occurs. However, other dosage regimens may be useful. The progress of the therapy is monitored easily by conventional techniques and assays. An exemplary dosing regimen is disclosed in WO 94/04188. The specification for the dosage unit forms of the invention is dictated by and directly dependent on the unique characteristics of the active compound and the particular therapeutic effect to be achieved and the limitations inherent in the art of compounding such an active compound for the treatment of individuals.

The pharmaceutical compositions can be included in a container, pack or dispenser together with instructions for administration.

The pharmaceutical compositions of interest can be administered alone, or can be administered in combination with one or more other pharmaceuticals, such as an oxime.

The nucleic acids encoding the bioscavenger molecules of interest can be delivered to the tissues most likely to be exposed to a toxic material or which are know to be susceptible to a toxic material. Thus, a vector can be administered subcutaneously or intradermally for skin exposures, or be delivered by a transdermal patch, for example. For aerosolized toxic agents, the nucleic acids of interest can be administered in a mouth wash, an inhaler and so on. For the inhaler, the nucleic acids of interest can be delivered as a liquid, a powder and so on, as known in the art.

The invention now will be exemplified in the following non-limiting examples.

The human BChE gene was kindly provided by Dr. Carolyn Chambers (WRAIR). The MaBChE gene was cloned and sequenced from the livers of two separate macaques using DNA oligonucleotide primers which were synthesized based on the HuBChE sequence. The sequencing revealed three amino acid differences between the 2 macaque sequences at 237 (tyrosine vs. phenylalanine), 261 (aspartic acid vs. glutamic acid) and 540 (glutamic acid vs. alanine) and 4% (23 aa out of 574 aa) differences from a human gene. As mentioned, rBChE is comprised of monomers, dimers and tetramers. Monomer BChE will be expressed. Tetramerization of monomers can be performed in vitro, optionally using the PRAD protein.

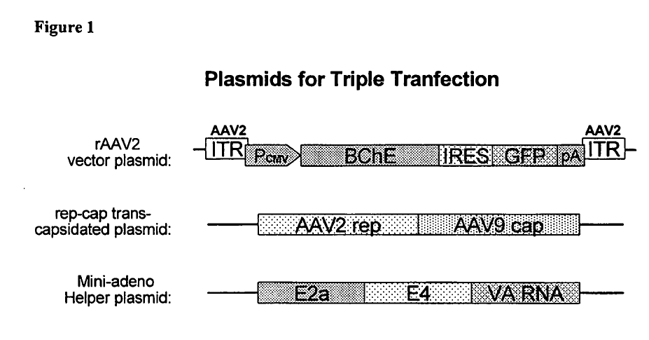

The small genome of AAV consists of two genes (rep and cap) enclosed by identical ITR sequences, with a total size of ˜4.7 kb. Thus, the size limit of the gene/s to be transferred is up to 4.7 kb. Both BChE (1809 nucleotides) and EGFP (805 nucleotides) are expressed using an internal ribosomal entry site (IRES) element (581 nucleotides) with a CMV promoter (588 nucleotides) and SV40 (222 nucleotides) or BGH (323 nucleotides) poly(A) signals. The elements above are engineered in one fragment in the order as shown in FIG. 1. The final single fragment (˜4 kb) will also have appropriate linkers at its 5′ and 3′ ends for insertion into rAAV vector plasmid.

Viral recombinants used for transduction are produced using the triple transient transfection system of Xiao at al (39). This involves the transfection of an rAAV2 vector plasmid, an AAV2 rep and AAV9 cap gene containing plasmid, and a helper plasmid containing adenoviral E2a, E4, and VA RNA genes into 293 cells containing adenoviral E1. Such a system totally eliminates the possibility of adenoviral protein contamination and contributes to the safety of gene delivery.

It should be noted that the addition of the GFP protein is to facilitate the visualization of infected cells.

Purified AAV particles carrying the inserts of interest are made practicing methods known in the art. AAV2/9 is purified by CsCl sedimentation.

The HuBChE and MaBChE containing viral particles are used to transduce both the IB3 airways epithelial cell line and the muscle C2C12 cell lines. Initially, titrations of the virus (1-1000 particles) are done to find optimal conditions. Routine monitoring for both enzyme activity in the supernatant and the presence of the transgene by EGFP fluorescence are done for ˜three months.

IB3-1 cells are undifferentiated simian virus 40 T antigen-transformed human lung epithelial cells isolated from a cystic fibrosis patient. IB3-1 cells are obtained from ATCC (JHU-52) and maintained on LHC-8 medium (Invitrogen) with 5% fetal bovine serum supplemented glutamine, 100 U/ml penicillin and 100 ug/ml streptomycin (P/S) (all from Invitrogen). C2C12 is an undifferentiated mouse muscle cell line. The cells are purchased from the ATCC (#CRL-1772) and cultured in Dulbecco's modified Eagle Medium (DMEM) plus 10% FCS and P/S. For cell differentiation 10% horse serum is used.

All experiments are performed in triplicate. Approximately 2×10⁵/well of IB3-1 and C2C12 cells are plated in 6-well plates and incubated in a 37° C. CO₂ incubator. Twenty four hours later, the culture medium is replaced with 0.5 ml of the appropriate serum-free media containing viral vectors at 1, 10, 100 and 1000 multiplicities of infection (MOI). After 2 hours incubation in a 37° C. CO₂ incubator, FCS is added to the media. The cells are examined for EGFP expression by fluorescent microscopy at any time after transduction.

The transduced cells are maintained for at least 3 months with weekly passages (split 1:4) and biweekly media changes. BChE activity is assayed daily for the first 3 days post transduction, then at the time of media changes and passages. The cells left after each passage are examined for the presence of EGFP using a fluorescence microscope or by FACS analysis of EGFP expression (at Virion Systems) to provide information on the percentage of infected cells at each MOI and the duration of expression. These analyses indicate the optimal MOI for larger scale transduction of each cell type. Number of transgene copies in the transduced cells 3 days after transduction will be determined by quantitative PCR analysis of rAAV genome sequences with suitable primer pairs targeting a vector site.

BChE activity was determined with the Ellman method (41) with butyrylthiocholine iodide (1 mM) as substrate in 0.1M sodium phosphate buffer, pH 7.0 at 25.0° C. Units of activity are umol of butyrylthiocholine hydrolyzed per minute.

Larger scale transduction (100 mm culture plate or T75 flask) proportionally use the same conditions as the initial conditions with respect to cell numbers, viral particle number and volume. To maximize BChE production, transduced cells are maintained with biweekly collection of supernatant without passage and the BChE supernatant purified as below.

Briefly, culture supernatant was clarified by centrifugation. The purification of BChE from supernatant was conducted essentially as described (43) although some differences in the methods of purification of human and macaque BChE exist. Supernatant was centrifuged at 10,000 rpm for 30 min using a GSA rotor and diluted 1:1 with 50 mM sodium phosphate, pH 8.0, and loaded onto either a 100 ml (small batch) and ˜800 ml (large batch) procainamide-Sepharose 4B affinity gel columns. After extensive washing with 0.2 M NaCl in 50 mM sodium phosphate, pH 8.0, enzyme was eluted with 0.2 M procainamide or NaCl in 50 mM sodium phosphate, pH 8.0. Fractions containing enzyme activity optionally were further purified on another 100 ml procainamide-Sepharose column.

Monosaccharide composition of the total carbohydrate, galactose and sialic acid content of the rBChE produced in vitro were analyzed (Glyco, CA) and compared to the native form. Analysis of monomers, dimers and tetramers was done using sucrose gradient analysis.

Due to the 1:1 stoichiometry between the enzyme and OP (42) and to rapid irreversible inhibition of OP-inhibited ChEs, protection against OP poisoning requires large amount of scavenger (predicted to be 200 mg/70 kg (−3 mg/kg) of circulating HuBChE for protection of humans against 0.9 and 1.0 LD50 of VX and soman respectively).

The pharrnacokinetics and immunological consequences of administration of purified homologous MaBChE into macaques at a dose similar to that required to prevent OP toxicity were investigated. The results (3) indicated very good stability in the plasma without any induction of anti-BChE antibody or adverse effects. Thus an iv injection of 7,000 U of homologous enzyme demonstrated much longer retention times in plasma (MRT=225±19 h) compared to those reported for heterologous HuBChE (33.7±2.9 h). A smaller second injection of 3,000 U given four weeks later also attained predicted plasma enzyme activity, but surprisingly, the MRT in the four macaques showed wide variation and the MRT ranged from 54 to 357 h. These results bode well for the potential use of HuBChE as a detoxifying drug in humans and this homologous nonhuman primate model will be used to provide the appropriate preclinical data.

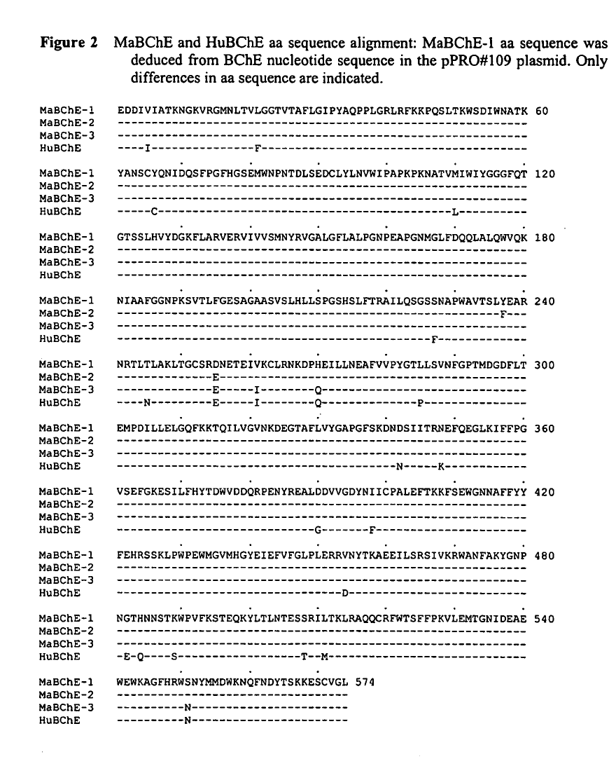

MaBChE (1,809 nucleotides) was amplified in two fragments (5′-fragment and 3′-fragment) which join at a convenient XbaI restriction site native to the BChE sequence. (FIG. 2).

A mammalian cell expression vector plasmid which transiently expresses MaBChE gene under the control of a CMV promoter was constructed (pPRO#109). Successful expression of MaBChE in both CHO-K1 and COS-7 cells was obtained. One day before transfection, 1.25×10⁵ CHO-K1 and 1.00×10⁵ COS-7 cells/well were plated in triplicate in 24-well plates and incubated in a 37° C., CO₂ incubator. Immediately before transfection, the media in each well was replaced with 0.2 ml OptiMEM and the cells in each well were transfected by the addition of transfection media containing 50 ul of OptiMEM, 1.5 ug pPRO#109, 4 ul Plus Reagent and 2 ul Lipofectamine Reagent. After 3 hours, the transfection media was replaced with 0.5 ml fresh culture media containing fetal calf serum (FCS). The BChE activities in the supernatant were assayed at 1, 2, 3, and 7 days after transfection. The supernatant was changed with complete media at 3 days after transduction. The activity at each time point represents the accumulated enzyme activity/ml from each well. Surprisingly, the macaque MaBChE gene was not expressed well in the macaque COS-7 line.

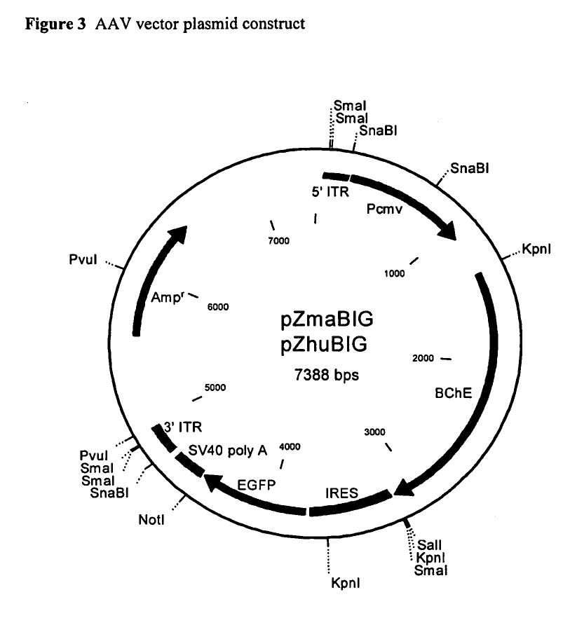

The human BChE cDNA including signal peptide sequence was PCR-amplified with 5′ and 3′ primers which contain KpnI and SalI restriction sites, respectively. An AAV vector plasmid, pZac2.1, obtained from Dr. Guang-Ping Gao (U. Penn) bears 5′- and 3′-AAV2 ITR's and an expression cassette comprising the CMV promoter and SV40 poly sequences. The amplified HuBChE cDNA was cloned into pZac2.1 at the unique KpnI and SalI sites (pZac2.1-HuBChE). HuBChE nucleotide sequence in the cloned vector plasmid was confirmed by direct DNA sequencing. Out of five clones, one showed authentic HuBChE sequences, which was used for final vector construction. IRES-EGFP fragment in the commercially available pEGFP2 plasmid was excised with SalI and NotI restriction enzymes and cloned into both pZac2.1-MaBChE and pZac2.1-HuBChE at corresponding SalI and NotI sites. The resulting pZac2.1-MaBChE-IRES-EGFP named as ‘pZmaBIG’ and pZac2. 1-HuBChE-IRES-EGFP ′pZhuBIG′(FIG. 3). Both vector plasmids nest a bicistronic cassette of BChE and EGFP between AAV2 5′-ITR and -3′ITR sequences.

As mentioned, the AAV2/9 virus was chosen because this serotype exhibits tropism for both muscle and lung; sites of BChE exposure and damage. Thus, several groups of C57/BL mice (10 per group) are used to assess the persistence of AAV in each of these tissues and the level of BChE produced both locally and systemically. Mice receive the high number of 5×10¹⁰ to 1×10¹¹ genome copies either by an intramuscular injection in 50 ul in the anterior tibialis muscles, intranasally, exposure by intrapulmonary (i.pl) delivery following a midline neck incision via the trachea or by both im and ip routes.

Tissues from animals are harvested by needle biopsy or at necropsy. Muscle or lung tissues (25-50 mg) from the rAAV administered animals are cut into small pieces, weighed then incubated with 2 mg/ml proteinase K in lysis buffer at 60° C. for one hours or until the tissue is completely lysed. Cultured cells (10⁴ -106 cells) are incubated in 2 mg/ml proteinase K in 200 ul PBS at 65° C. for 10 min. The lysate is centrifuged and DNA is quantified using Hoechst 33258 Dye. To evaluate rAAV persistence in the tissues or cultured cells, the lysate is subjected to Real Time PCR with a pair of primers synthesized based on the DNA sequence of rAAV2 vector. A 5′-primer sequence will be based on the CMV promoter DNA sequence and 3′-primer on the MaBChE cDNA sequence. Recombinant AAV vector plasmid will be used as the standard.

Blood samples (5 μl diluted into 95 μl water) will be taken by tail nick at designated time points for MaBChE levels using the Ellman assay. Hematocrit levels are measured in these animals by microcapillary centrifugation. Bronchoalveolar lavage is performed using two aliquots of 500 μl of sterile PBS instilled through a 21-gauge catheter into the trachea after exposure via a midline neck incision. The fluid is immediately aspirated and the aliquots pooled for analysis.

At various days following vector administration, animals receiving the AAV2/9 particles were sacrificed, and their lungs were inflated with OCT compound (Sakuraus Finetek USA Inc., Torrance, Calif., USA), frozen, cryosectioned, and stained for protein expression (26). In the readministration experiment, lungs were harvested 28 days after vector delivery and homogenized. Protein concentration in each sample was standardized to 2 mg/ml before measurement of the galactosidase concentration using a commercially available ELISA (Roche Molecular Biochemicals, Mannheim, Germany) according to the manufacturer's instructions. In the experiments involving alkaline phosphatase expression, animals were euthanized 28 days after vector administration, at which point the left lung was immediately homogenized in lysis buffer (Reporter Gene Assay Lysis Buffer; Roche Molecular Biochemicals, Indianapolis, Ind., USA) containing protease inhibitor (COMplete Protease Inhibitor Cocktail; Roche Molecular Biochemicals, Indianapolis, Ind., USA). The homogenate was centrifuged to remove tissue debris, and the supernatant was stored for analysis. The right lung and trachea were embedded in OCT compound, frozen, and cryosectioned. The slides were fixed with 0.5% glutaraldehyde and incubated in 1 mM MgCl₂ at 65° C. to inactivate endogenous alkaline phosphatase. After rinsing, the slides were stained with alkaline phosphatase buffer (1.2 g Tris-base, 0.584 g NaCl, 1.017 g MgCl₂ in 100 ml water, pH 9.5), nitro-blue tetrazolium chloride, and 5-bromo-4-chloro-3-indolylphosphate toluidine. Sections were incubated for 30 minutes, rinsed, and counterstained with Nuclear Fast Red. The lung homogenate was quantitatively previously for placental alkaline phosphatase using two separate methods according to the manufacturers' protocols (SEAP Reporter Gene Assay, Roche Molecular Biochemicals, Indianapolis, Ind., USA; and Innotest hPALP ELISA, Innogenetics, Ghent, Belgium).

MaBChE produced in mice and macaque by AAV2/9 delivery is analysed for carbohydrate composition, for example, by GlycoSolutions (MA) to determine the forms in blood, lung and muscle. The first sugar analysis (using about 240 ug protein) measures the amounts of neutral monosaccharides, such fucose, N-acetylglucosamine, N-acetylgalactosamine, mannose and galactose) in samples. The second assay measures two sialic acids (N-acetylneuraminic acid (NeuAc/NANA) and N-glycolylneuraminic acid (NeuGc/NGNA) in samples. Sialic acids are separately from neutral monosaccharides because they are more acid labile and are destroyed by the acid hydrolysis conditions required to release neutral monosaccharides from the protein. Analysis of the recombinant is performed both before and after chemical modification.

Samples are treated in acid to release the monosaccharides. The monosaccharides are then separated isocratically in 22 mM NaOH by HPAEC (high pH anion-exchange chromatography) using a CarboPac PA1 column (Dionex, Sunnyvale, Calif.) and detected by PAD (pulsed amperometric detection) using an electrochemical detector. This chromatography is performed using a Dionex BioLC. The monosaccharides are quantified relative to a standard curve of each standard. The standard curve is obtained under the same conditions and at the same time as the samples.

Samples containing 90 μg are hydrolyzed in mild acid to release sialic acids. The sialic acids are then separated using a gradient from 50 to 250 mM sodium acetate in 100 mM NaOH over 15 minutes using HPAEC (high pH anion-exchange chromatography) and a CarboPac PA1 column (Dionex, Sunnyvale, Calif.) and detected by PAD (pulsed amperometric detection). The chromatography is performed using a Dionex BioLC. The sialic acids are quantified relative to a standard curves of each sialic acid. The values usually are expressed as mol sialic acid/mol protein.

Plasma is collected as small aliquots and pooled for purification. In a previous monkey study, 6 litres of frozen plasma (containing about 48,000 U) yielded about 44,000 U (˜7,300 U per litre, 92% recovery) after purification. Briefly; frozen pooled plasma is allowed to thaw at 4-8° C. and then is clarified by centrifugation (10,000 rpm) or filtering through cheese cloth. The purification of MaBChE from plasma is conducted essentially as described (47). The plasma is centrifuged at 10,000 rpm for 30 min using a GSA rotor and diluted 1:1 with 50 mM sodium phosphate, pH 8.0, and loaded on either 100 ml (small batch) and 800 mL procainamide-Sepharose 4B affinity gel columns (16). After extensive washing with 0.2 M NaCl in 50 mM sodium phosphate, pH 8.0, enzyme is eluted with 0.2 M procainamide or NaCl in 50 mM sodium phosphate, pH 8.0. Fractions containing enzyme activity are further purified on the 100 ml procainamide-Sepharose column. To ensure sterility prior to injection, the material is then dialyzed against the same sterile buffer using 12,000 to 14,000 molecular weight cut off dialysis membrane and exposed to UV using a transilluminator while in the dialysis membrane.

Nine macaques are used with homologous MaBChE gene to provide protection against nerve toxins. Monkeys (three per group) will receive a high dose of 10¹²-10¹³ genome copy per kilogram by
(i) Intramuscular injection (four injections in each of four limbs equaling 16 injections at 100 ul each);
(ii) intrapulmonary administration via an endotracheal tube; and
(iii) Intramuscular injection plus intrapulmonary administration.

MaBChE activity is measured in blood and tissues similar to the described in the mice studies. Monkeys initially are bled every week for two months. In those that were given the AAV2/9 vector im muscle needle biopsies are taken every 14 days at the marked injection sites. The tissue is homogenized and AAV persistence is measured by PCR. Monkeys are anaesthetized with ketamine prior to all injections. Monkeys are intubated and the AAV is instilled through a cuffed 2.5-3 Fr gauge endotracheal tube.

Human and macaque BChE in serum are composed predominantly of tetramers with the tetramerization domain being located within the last 40 C-terminal residues of each monomeric subunit (534-574) (13). The insertion of a stop codon at G534 results in a monomeric form lacking 41 C-terminal residues that is incapable of oligomerization. Both the truncated and wild type MaBChE genes have been cloned and expressed in CHO K1 cells using the pcDNA vector containing a CMV promoter. Tetramerization of the recombinant WT monomeric BChE molecules was observed either by co-expression with the N-terminus of COLQ gene which includes the proline-rich attachment domain (PRAD) (14) or by the addition of PRAD to the culture medium.

Previously, PRAD has been shown to increase the amount of tetrameric BChE from 10%-70%. Co-transfection with the PRAD gene increased expression and the number of positive BChE-producing clones. The results from the experiment indicate that the CHO K1 cells transfected with the truncated MaBChE gene yielded 23/48 MaBChE producing clones when co-transfected with PRAD and 6/42 in the absence of PRAD.

To show that the WT and truncated MaBChE genes can successfully encode bioactive tetrameric and monomeric proteins when expressed in CHO-K1 cells, purified proteins were subjected to 5-20% sucrose gradients (centrifugation was carried out using an SW41 Ti rotor (Beckman) at 30,000 rpm for 18 hrs at 4° C.). More than 90% of the WT gene produced tetrameric MaBChE and as expected all of the truncated gene produced monomeric enzyme.

### BIBLIOGRAPHY

1. Doctor, B P., Maxwell, D M., Ashani,Y., Saxena, A. and Gordon, R K. 2001. Somani, S M and Romano, J A, Eds. CRC Press, New York, p191-214.

2. Lenz, D E., Broomfield, C A., Maxwell, D M., Cerasoli, D M. 2001. Somani, S M and Romano, J A, Eds. CRC Press, New York, p215-243.

3. Rosenberg, Y J., Luo, C., Ashani, Y., Doctor, B P., Fischer, R., Wol fee, G., Saxena, A. 2002. Life Sciences 72:124-134

4. Cascio, C., Comite, Ghiara. M., Lanza, G., Ponchione. A. 1988. Minerva Anestesiol. 54:337-338.

5. Bellon, G, Michel-Calemard L, and 24 more authors. 1997. Hum Gene Ther. 8, 15-25.

6 Gunderson C H., Lehman. C., Sidell, F R., Jabbari B. 1992. Neurology 42; 946-50.

7. Wolthuis, O L., Berends, Meeter, E. 1981. Fund. Appl. Toxicol. 1:183-192.

8. Sidell, F R. 1974. Clin. Toxicol. 7:1-17.

9. Wolfe A D, Rush R S, Doctor B P, Koplovitz I, Jones D. 1987. Fundam Appl Toxicol. 9:266-70.

10. Raveh, L., Grunwald, J., Marcus, D., Rapier, Y., Cohen, E., and Ashani, Y. 1993. Biochem Pharmacol 45, 2465-2474.

11. Ashani, Y., Grunwald, J., Graur, E., Brandeis, R., Cohen, E., Raveh, L. 1993. Med Defense Bioscience Rev 3:1025-1034.

12 Doctor, B P., Raveh, L., Wolfe, A D., Maxwell, D M., Ashani, Y. 1991. Neurosci Biobehav Rev 15:123-8.

13. Broomfield, C A., Maxwell, D M., Solana, R P., Castro, C A., Finger, A V. And Lenz, D E. 1991. J. Pharnacol. Exper. Therapeut. 259, 633-640.

14. Lockridge, O. 1990. Pharrnacol Ther 47, 35-60.

15. Saxena, A., Ashani, Y., Raveh, L., Stevenson, D., Patel, T., Doctor, B P. 1998. Mol Pharmacol 53, 112-22.

16. Velan, B., Kronen. C., M.arkus, D., Ordlentlich, A., Motola, L., Zeligrer. N., Reuveny. S., and Shafferman. A. 1996. Medical Defense Science Review. 1:203-212.

17. Mesulam M M, Guillozet A, Shaw P, Levey A, Duysen E G, Lockridge O. 2002. Neuroscience. 110:627-39.

18. Saxena A., Ashani Y., Raveh L., Stevenson D., Patel, T., Doctor B P. 1998. Mol. Pharmacol., 53:112-122.

19. Blong R M, Bedows E, Lockridge O. 1997. Biochem J., 327:747-57.

20. Buning H, Nicklin S A, Perabo L, Hallek M, Baker A H. 2003. Curr Opin Mol Ther. 5:367-75.

21. Hildinger M, Auricchio A, Gao G, Wang L, Chirmule N, Wilson J M. 2001. J Virol. 75(13):6199-203.

22. Gao G P, Alvira M R, Wang L, Calcedo R, Johnston J, Wilson J M. 2002. Proc Natl Acad Sci USA. 99:11854-9.

23. Tenenbaum L, Lehtonen E, Monahan P E. 2003. Curr Gene Ther. 3:545-65.

24. Erles K, Sebokova P, Schlehofer J R. 1999. J Med Virol. 59:406-11.

25. Daly T M. 2004. Methods Mol Biol. 246:157-65.

26. Xiang Z Q, Gao G P, Reyes-Sandoval A, Li Y, Wilson J M, Ertl H C. 2003. J Virol. 77:10780-9.

27. Fitzgerald J C, Gao G P, Reyes-Sandoval A, Pavlakis G N, Xiang Z Q, Wlazlo A P, Giles-Davis W, Wilson J M, Ertl H C. 2003. J Immunol. 170:1416-22.

28. Gao G, Vandenberghe L H, Alvira M R, Lu Y, Calcedo R, Zhou X, Wilson J M. 2004. J Virol, 78:6381-6388.

29. Arruda V R, Schuettrumpf J, Herzog R W, Nichols T C, Robinson N, Lotfi Y, Mingozzi F, Xiao W, Couto L B, High KA. 2004. Blood. 103:85-92.

30. During, M. J., Samulski, R. J., Elsworth, J. D., Kaplitt, M. G., Leone, P., Xiao, X., et al. 1998. Gene Ther. 5:820-827.

31. Lai C M, Yu M J, Brankov M, Barnett N L, Zhou X, Redmond T M, Narfstrom K, Rakoczy P E. 2004. Genet Vaccines Ther. 2:3.

32. Snyder, R. O., Miao, C., Meuse, L., Tubb, J., Donahue, B. A., et al. 1999. Nat. Med. 5, 64-70.

33. Xu Y, Gu Y, Wu P, Li G W, Huang L Y. 2003. Hum Gene Ther. 14:897-906.

34. Moss R B, Rodman D, Spencer L T, Aitken M L, Zeitlin P L, Waltz D, Milla C, Brody A S, Clancy J P, Ramsey B, Hamblett N, Heald A E. 2004. Chest. 125:509-21.

35. Tan, M., Qing, K., Zhou, S., Yoder, M. C., and Srivastava, A. 2001. Mol. Ther. 3, 940-946.

36. Zabner J, Seiler M, Walters R, Kotin R M, Fulgeras W, Davidson B L, Chiorini J A. 2000. J Virol. 74(8):3852-8.

37. John F. Engelhardt. 2002. J. Clin. Invest. 110:429-432.

38. Flotte T R, Brantly M L, Spencer L T, Byrne B J, Spencer C T, Baker D J, Humphries M. 2004. Hum Gene Ther. 15:93-128.

39. Xiao X, Li J, Samulski R J. 1998. J Virol. 72:2224-32.

40. Rabinowitz J E, Bowles D E, Faust S M, Ledford J G, Cunningham S E, Samulski R J. 2004. J Virol. 78:4421-32.

41. Ellman, G L., Courtney, K D., Andres, V., Feathstone, R M. 1961. Biochem. Pharmacol. 7:88-95

42. Raveh L, Grauer E, Grunwald J, Cohen E, Ashani Y. 1997. Toxicol Appl Pharmacol. 145:43-53.

43. Grunwald J, Marcus D, Papier Y, Raveh L, Pittel Z, Ashani Y. 1997. J Biochem Biophys Methods. 27; 34:123-35.

44. Weitzman M D, Kyostio S R, Kotin R M, Owens R A. 1994. Proc Natl Acad Sci USA 91:5808-12.

Additional features and advantages of the present invention are described in, and will be apparent from, the above detailed description of the invention and the appended claims.

It should be understood that various changes and modifications to the presently preferred embodiments described herein will be apparent to those skilled in the art. Such changes and modifications can be made without departing from the spirit and scope of the present invention and without diminishing its intended advantages.

I claim:

1. A method for inactivating a toxic agent comprising administering to a host in need of treatment a therapeutically effective amount of a nucleic acid encoding a detoxifying agent or variant thereof that inactivates said toxic agent.

2. The method of claim 1 comprising a viral vector.

3. The method of claim 2, wherein said viral vector is obtained from adenovirus, adeno-associated virus (AAV) or HIV.

4. The method of claim 1 wherein said administering comprises delivering said nucleic acid to skin or lung.

5. The method of claim 1 wherein said detoxifying agent is butyrylcholinesterase (BChE), acetycholinesterase (AChE), organophosphate hydrolases (OPH), organophophorous acid anhydride hydrolases (OPAA), parathion hydrolase, paraoxonase or carboxylesterase.

6. The method of claim 1 wherein said toxic agent is an insecticide or an organophosphate (OP) nerve agent.

7. The method of claim 6, wherein said nerve agent is VX (ethyl-S-2-diisopropylaminoethyl-phosphano-thiolate), MEPQ (7-(methylethoxy-phosphinyloxy)-1-methylquinolinium iodide), soman (pinacolyl methlphosphonofluoridate), or DFP (diisopylfluorophosphate).

8. The method of claim 1 wherein said therapeutically effective amount is a level sufficient to protect against 2 LD₅₀ of a toxic agent.

9. The method of claim 1 wherein said administering occurs prior to toxic agent exposure.

10. The method of claim 1 wherein said host is livestock or a domesticated animal.

11. A method of making a detoxifying agent comprising expressing a nucleic acid expressing said detoxifying agent in a mammalian cell or a plant cell.

12. The method of claim 11, wherein said detoxifying agent is BChE.

13. The method of claim 12, wherein said BChE is expressed as a monomer that forms a tetramer.
